(19) United States
(12) Patent Application Publication
(10) Pub. No.: US 2024/0047051 A1
(43) Pub. Date: Feb. 8, 2024

(54) EMERGENCY BLOOD DISPATCHING METHOD AND SYSTEM BASED ON EARLY PREDICTION AND UNMANNED FAST DELIVERY

(71) Applicant: ZHEJIANG LAB, Hangzhou (CN)

(72) Inventors: Jingsong LI, Hangzhou (CN); Jing XIA, Hangzhou (CN); Yinghao ZHAO, Hangzhou (CN); Yu TIAN, Hangzhou (CN); Tianshu ZHOU, Hangzhou (CN)

(21) Appl. No.: 18/353,865

(22) Filed: Jul. 17, 2023

(30) Foreign Application Priority Data
Aug. 2, 2022 (CN) 202210921805.4

(51) Int. Cl. G16H 40/20; G16H 50/20; B64U 10/00

(52) U.S. Cl. CPC G16H 40/20; G16H 50/20; B64U 10/00; G06Q 50/28

(57) ABSTRACT

Disclosed is an emergency blood dispatching method and system based on early prediction and unmanned fast delivery. In the present disclosure, an emergency blood use prediction model and an unmanned aerial vehicle fast delivery route are introduced, blood use demands of pre-hospital emergency trauma patients are accurately predicted, pre-hospital emergency blood transfusion of patients is achieved through unmanned aerial vehicle sites, it does not need to consume a lot of road traffic time to arrive at a hospital and then starts blood transfusion, the speed of blood supply and treatment quality of the patients with massive traumatic hemorrhage are improved, and it is of great value to rescue remote mountain trauma patients. The present disclosure evaluates blood use demands of the hospital in real time, and combines an unmanned aerial vehicle and a blood delivery car to fast deliver needed blood products from a blood center to the hospital.

### CROSS-REFERENCE TO RELATED APPLICATIONS

The present application claims priority to Chinese Patent Application No. 202210921805.4, filed on Aug. 2, 2022, the content of which is incorporated herein by reference in its entirety.

### TECHNICAL FIELD

The present disclosure belongs to the technical field of medical information and unmanned aerial vehicle, in particular to an emergency blood dispatching method and system based on early prediction and unmanned fast delivery.

### BACKGROUND

At present, the pre-hospital first aid method for patients with severe trauma is to transport the patients to the hospital first, and then judge the blood demand of the patients and comprehensively evaluate the blood supply and demand of the hospital after the patients arrive at the hospital. When necessary, a rescuer will apply for invoking blood from the blood center, and transport blood products through road traffic. The existing problems in the related art is that the response to emergency blood is not fast enough, which are embodied in the following aspects: (1) the blood supply efficiency is low for patients with traumatic hemorrhage, especially for patients in remote mountainous areas; (2) if a large-scale traumatic event occurs, the speed of emergency blood supply to the hospital is slow.

At present, unmanned aerial vehicles have been tried to be used in the medical field. The unmanned aerial vehicles have been applied to assist the transportation of daily blood products, but not to emergency blood supply. In the first-aid process of patients with severe trauma, the efficiency of emergency blood supply is still insufficient.

### SUMMARY

In view of the above technical problems, the present disclosure provides an emergency blood dispatching method and system based on early prediction and unmanned fast delivery. The present disclosure uses unmanned flight dedicated lines to improve emergency blood supply efficiency and treatment quality, which is embodied in the following:
(1) For he traumatic bleeding event in remote mountainous areas, the present disclosure uses the emergency blood use prediction model and the unmanned aerial vehicle route from the hospital to the unmanned aerial vehicle site to realize the emergency blood transfusion of patients at the pre-hospital unmanned aerial vehicle site, and does not need to spend a lot of road traffic time to arrive at the hospital before starting blood transfusion.
(2) In view of large-scale trauma events, the blood consumption surges, and the hospital's blood inventory is insufficient, so it was necessary to quickly adjust blood to the blood center; the present disclosure realized the real-time evaluation of the hospital's emergency blood supply and demand and the fast replenishment of blood products by unmanned aerial vehicles by using the emergency blood use prediction model and the unmanned aerial vehicle route from the blood center to the hospital.

The purpose of the present disclosure is achieved through the following technical solutions:

According to a first aspect of this specification, there is provided an emergency blood dispatching method based on early prediction and unmanned fast delivery; the method includes the following steps:
Step 1, collecting pre-hospital trauma patient samples, and building a staged multi-level emergency blood use prediction model;
Step 2, predicting a blood use demand of a patient based on the emergency blood use prediction model according to trauma patient information;
Step 3, using a two-layer structure weighted composite ratio algorithm to realize intelligent recommendation of a transport destination of the patient and pre-hospital blood delivery through a comparative evaluation with an injury point as a circle center and a weighted triangle comprehensive evaluation according to a location of the patient, and a distance of the patient from surrounding unmanned aerial vehicle sites and surrounding hospitals, to assist an emergency doctor in decision making;
Step 4, counting up total demands of blood products in each hospital, calculating a demand tension degree for all blood products of all patients in each hospital and performing ranking, so as to form an in-hospital blood product supply sequential order table;
Step 5, ranking a priority of the unmanned aerial vehicles, comparing a difference between the unmanned aerial vehicles and blood delivery cars and adjusting indefinite-length route sequences with a goal of minimizing waiting time constantly and cyclically according to the total demands and a supply tension degree of the blood products in each hospital, an in-hospital inventory, and a number of in-transport blood products based on a circulation sequence algorithm combining the unmanned aerial vehicles and the blood delivery cars, so as to realize intelligent dispatching of transport tools and fast delivery of the blood products; and
Step 6, evaluating a supply and demand relationship of the blood products, blood use conditions of all the patients, and states of all the transport tools of each hospital in real time, evaluating whether a current dispatching and delivery scheme meets a demand, and if the scheme does not meet the demand, updating the dispatching and delivery scheme.

Further, the Step 1 is specifically as follows:

Collecting the pre-hospital trauma patient samples, and recording pre-hospital and in-hospital multidimensional information; a prediction target Y being a category k, and selecting a preliminary scheme or an improved scheme according to an emergency degree.

In the preliminary scheme, K is valued as 2, and the prediction target Y is 24-hour red blood cell infusion volume belonging to [0, 4] or (4, +∞), valued as 0 and 1 respectively; if Y=0, emergency blood use is not applied; if Y=1, a blood use for 2 units of O-type red blood cells is applied at a scene of injury immediately.

In the improved scheme, K is valued as 3, and the target Y is predicted to be 24-hour red blood cell infusion volume belonging to 0 or (0, 4] or (4, +∞), valued as 0, 1 and 2 respectively; if Y=0, blood transfusion is not needed; if Y=1, a blood type is measured after arriving at a hospital, and then a blood use for 2 units of specific blood-type red blood cells is applied; and if Y=2, a blood use for 2 units of O-type red blood cells is applied at the scene of injury immediately, and the blood type is measured after arriving at the hospital, and then a blood use for 2 units of specific blood-type red blood cells is applied.

The staged multi-level emergency blood use prediction model is represented as:

$$[ {\hat{y}}_{final}^{0} , … , {\hat{y}}_{final}^{k} , … , {\hat{y}}_{final}^{K - 1} ] = ( 2 - s ) * {f}_{1} ( {X}_{p r e} ) + ( s - 1 ) * {f}_{2} ( [ {X}_{pre} , {X}_{i n} ] )$$

$$\hat{Y} = {arg max}_{k} {\hat{y}}_{final}^{k}$$

where s represents a prediction stage, s=1 represents a pre-hospital stage, s=2 represents an in-hospital stage, functions f₁ and f₂ respectively represent a pre-hospital prediction model and an in-hospital prediction model, Xₚᵣₑ, Xᵢₙ respectively represent a pre-hospital feature set and an in-hospital newly-added feature set after mean value vacancy filling and normalization pretreatment; [Xₚᵣₑ, Xᵢₙ] represents that Xₚᵣₑ, Xᵢₙ are spliced, ŷ_{final}^{k} is a prediction value of the category k output by the staged multi-level emergency blood use prediction model, ŷ_{final}^{k} is valued as [0,1], Ŷ is a predicted blood use category, Ŷ in the preliminary scheme is valued as 0 or 1, and Ŷ in the improved scheme is valued as 0 or 1 or 2.

Further, in the staged multi-level emergency blood use prediction model,

[ŷₚᵣₑ⁰, . . . , ŷₚᵣₑ^{k}, . . . , ŷₚᵣₑ^{K-1}]=f₁(Xₚᵣₑ)=softmax(W₁·Xₚᵣₑ+b₁)

[ŷᵢₙ⁰, . . . , ŷᵢₙ^{k}, . . . , ŷᵢₙ^{K-1}]=f₂([Xₚᵣₑ, Xᵢₙ])=softmax(W₂·[Xₚᵣₑ, Xᵢₙ]+b₂)

where softmax(⋅) represents a softmax function, W₁, W₂ represent trainable weight parameters, represents a matrix multiplication; b₁, b₂ represent trainable bias parameters, ŷₚᵣₑ^{k} is a prediction value of the category k output by the pre-hospital prediction model, ŷᵢₙ^{k} is a prediction value of the category k output by the in-hospital prediction model, K is valued as 2 or 3, and ŷₚᵣₑ^{k}, ŷᵢₙ^{k} are valued as [0,1]; when K is valued as 2, representing the preliminary scheme, and when K is valued as 3, representing the improved scheme.

A total loss function Lₜₒₜₐₗ is:

$${L}_{total} = α * {L}_{pre} + ( 1 - α ) * {L}_{i n}$$

$${L}_{pre} = \frac{1}{M} {\sum }_{i = 1}^{M} ( - {\sum }_{j = 0}^{K - 1} I ( {Y}_{i} = j ) ln {\hat{y}}_{pre}^{ij} ) + {λ}_{1} {‖ {W}_{1} ‖}_{2}$$

$${L}_{i n} = \frac{1}{M} {\sum }_{i = 1}^{M} ( - {\sum }_{j = 0}^{K - 1} I ( {Y}_{i} = j ) ln {\hat{y}}_{i n}^{ij} ) + {λ}_{2} {‖ {W}_{2} ‖}_{2}$$

where α is a weight coefficient, Lₚᵣₑ, Lᵢₙ are a pre-hospital prediction model loss function and an in-hospital prediction model loss function respectively, M is a sample size, I(⋅) is an indicator function, Yᵢ is a real category of an ith sample, ŷₚᵣₑ^{ij}, ŷᵢₙ^{ij} are prediction values of categories j of ith samples output by the pre-hospital prediction model and the in-hospital prediction model respectively, λ₁, λ₂ are penalty term coefficients, and ∥⋅∥₂ represents an L2 norm.

Aiming at minimization of Lₜₒₜₐₗ, optimal parameters of the staged multi-level emergency blood use prediction model are obtained by a gradient descent method.

Further, the Step 2 specifically includes:

For each trauma patient, inputting the pre-hospital information of the patient into the staged multi-level emergency blood use prediction model built in Step 1, and outputting an emergency blood use category of the patient; after the patient arrives at the hospital, inputting both the pre-hospital information and in-hospital information of the patient into the staged multi-level emergency blood use prediction model built in Step 1 to update an emergency blood use prediction result.

In the preliminary scheme, a prediction of 1 represents that emergency blood use is needed, that is, a blood use for 2 units of O-type red blood cell is applied at the scene of injury immediately; a prediction of 0 represents that emergency blood use is not needed.

In the improved scheme, a prediction of 2 represents that a demand for a red blood cell blood product is very emergent, that is, a blood use for 2 units of O-type red blood cells is applied at the scene of injury immediately, the blood type is measured after arriving at the hospital, and then a blood use for 2 units of specific blood-type red blood cells is applied; a prediction of 1 represents that the demand for the red blood cell blood product is in moderate emergency, that is, the blood type is measured after arriving at the hospital, and then a blood use for 2 units of specific blood-type red blood cells is applied; and a prediction of 0 represents that blood transfusion is not needed.

Further, the Step 3 is divided into the following two conditions:

Condition 1: for the patient predicted not to need the O-type red blood cells in Step 2, comparing road traffic time arriving at each hospital by taking the injury point as the circle center, suggesting transporting the patient to a hospital NHI with the shortest road traffic time for treatment, and a blood use demand of the patient corresponding to the hospital NHI.

Condition 2: for the patient predicted to need the O-type red blood cells in Step 2, determining to transport the patient to a certain unmanned aerial vehicle site to have O-type red blood cell emergency blood transfusion, then transport the patient to a nearby hospital for further treatment, or transport the patient to a certain hospital to have the O-type red blood cell emergency blood transfusion and further treatment, and each unmanned aerial vehicle site belonging to a hospital with the shortest unmanned aerial vehicle flight consumption time, which is specifically as follows.

(a) A shortest road traffic time TNH for transporting the patient from the scene of injury to the hospital by an emergency vehicle is calculated, and a hospital serial number NHI corresponding to the TNH is recorded.

(b) A shortest time TNS for transporting the patient from the scene of injury to the unmanned aerial vehicle site by the emergency vehicle for O-type red blood cell emergency blood transfusion is calculated, and an unmanned aerial vehicle site serial number NSI corresponding to the TNS is recorded.

(c) Weighed triangle comprehensive evaluation is performed on the hospital NHI and the unmanned aerial vehicle site NSI, and a weighed triangle judgment index C is calculated as follows:

$$C = \frac{T N H}{\sqrt{2 * T N S * ( T N S + 0.5 * T S H )}} - 1$$

where TSH is the road traffic time from the unmanned aerial vehicle site NSI to a hospital Q with shortest consuming time to the site.

If the index C is greater than 0, it is suggested to transport the patient to the unmanned aerial vehicle site NSI for O-type red blood cell emergency blood transfusion, then transport the patient to the hospital Q for further treatment, supply the blood use demand of the patient at the unmanned aerial vehicle site NSI by the hospital affiliated to the unmanned aerial vehicle site, and supply the blood use demand for further treatment by the hospital Q; and otherwise, it is suggested to transport the patient to the hospital NHI for O-type red blood cell emergency blood transfusion and further treatment, and the blood use demand of the patient corresponds to the hospital NHI.

Further, in Step 4, counting up the total demands of the blood products in each hospital specifically includes the following steps.

Recording a number of all patients in a hospital i at time t as Nᵢ, comprising patients transported to the hospital from the scene of injury or the unmanned aerial vehicle site, and patients having emergency blood transfusion at the unmanned aerial vehicle site managed by the hospital.

For the patient n, adopting the staged multi-level emergency blood use prediction model to predict a category Ŷₙ, and performing calculation to obtain the number Rₙ of red blood cell blood product demands of the hospital for the treatment of the patient n through Ŷₙ, a patient treatment route, and a patient blood type determination status.

In the preliminary scheme, if Ŷₙ=0, Rₙ=0; if Ŷₙ=1, determining whether the emergency blood product of the patient n is supplied by the hospital; if O-type red blood cell emergency blood transfusion is performed at the hospital or the unmanned aerial vehicle site managed by the hospital, Rₙ=2, and if the hospital is not required to prepare the emergency blood product of the patient n, Rₙ=0.

In the improved scheme, if Ŷₙ=0, Rₙ=0; if Ŷₙ=1, determining whether a blood type of the patient n has been determined at time t; if the blood type is not determined, Rₙ=0, if the blood type has been determined, Rₙ=2, if Ŷₙ=2, determining whether O-type red blood cells for emergency blood transfusion of the patient n are supplied by the hospital, whether the specific blood-type red blood cells for further treatment are supplied by the hospital, and whether the blood type of the patient n has been determined at time t are determined; if all the red blood cells of the patient n are supplied by the hospital and the blood typed is not determined, Rₙ=2, if all the red blood cells of the patient n are supplied by the hospital and the blood type has been determined, Rₙ=4; if for the patient n, only the O-type red blood cells are supplied by the hospital, Rₙ=2; if for the patient n, only the specific blood-type red blood cells are supplied by the hospital and the blood type is not determined, Rₙ=0; and if for the patient n, only the specific blood-type red blood cells are supplied by the hospital and the blood type has been determined, Rₙ=2.

Gathering the blood use demands of all the patients in the hospital, and evaluating total blood product demands at time t, wherein a total demand of the blood products of the hospital i at time t is Dᵢ=Σₙ₌₁^{N}^{ᵢ}Rₙ.

Further, in Step 4, calculating the demand tension degree of all the blood products of all the patients in each hospital and performing ranking, so as to form the in-hospital blood product supply sequential order table, specifically including the following steps.

For the patient n in the hospital i, adopting the staged multi-level emergency blood use prediction model to predict the category Ŷₙ, calculating a blood use tension degree zₙ^{(patient)} of the patient n in the hospital i in combination with a duration of the patient n waiting for the blood product, and calculating a demand tension degree z_{n,p}^{(blood)}, p=1, . . . , Pₙ of all red blood cells of the patient n in the hospital i according to zₙ^{(patient)}, where Pₙ is a total demand for the red blood cells of the patient n.

In the preliminary scheme, if Ŷₙ=0, zₙ^{(patient)}=0; if Ŷₙ=1, zₙ^{(patient)}=gₙ*AWTₙ, where gₙ represents whether the emergency blood product of the patient n is supplied by the hospital; if the blood product is supplied by the hospital, gₙ=1, otherwise, gₙ=0, AWTₙ represents the time spent by the patient n in waiting for the emergency blood product at time t; if Ŷₙ=0, no z_{n,p}^{(blood)}; and if Ŷₙ=1, the demand tension degree of the blood product is z_{n,p}^{(blood)}=zₙ^{(patient)}, p=1,2.

In the improved scheme, if Ŷₙ=0, zₙ^{(patient)}=0; if Ŷₙ=1, zₙ^{(patient)}=gₙ*AWTₙ, where gₙ represents whether the emergency blood product of the patient n is supplied by the hospital and whether the blood type has been determined; if the emergency blood product is supplied by the hospital and the blood type has been determined, gₙ=1, and otherwise gₙ=0, AWTₙ represents the time spent by the patient n in waiting for the emergency blood product at time t; if Ŷₙ=2, zₙ^{(patient)}=A*(gₙ⁽¹⁾*AWTₙ⁽¹⁾+γ*gₙ⁽²⁾*AWTₙ⁽²⁾), where A is a ratio coefficient of importance of blood transfusion for very emergent patients to importance of blood transfusion for moderate emergent patients, A>1, gₙ⁽¹⁾, gₙ⁽²⁾ represents whether the O-type red blood cell blood product for first emergency treatment of the patient n is supplied by the hospital, whether the specific blood-type red blood cells for further treatment are supplied by the hospital and whether the blood type has been determined respectively; if the O-type red blood cell blood product for first emergency treatment is supplied by the hospital, gₙ⁽¹⁾=1, otherwise gₙ⁽¹⁾=0; and if the specific blood-type red blood cells for further treatment are supplied by the hospital and the blood type has been determined, gₙ⁽²⁾=1, otherwise gₙ⁽²⁾=0, where AWTₙ⁽¹⁾, AWTₙ⁽²⁾ represents the time spent by the patient n in waiting for O-type red blood cells required for first emergency treatment at time t and time spent by the patient n in waiting for the specific blood-type red blood cells required for further treatment at time t respectively, γ is a value discount factor of the specific blood-type red blood cells required for further treatment, and γ∈[0,1); if Ŷₙ=0, no z_{n,p}^{(blood)}; if Ŷₙ=1 the demand tension degree for the blood product is z_{n,p}^{(blood)}=zₙ^{(patient)}, p=1,2; if Ŷₙ=2, the demand tension degree for the blood product is z_{n,p}^{(blood)}=A*gₙ⁽¹⁾*AWTₙ⁽¹⁾, p=1,2, but z_{n,p}^{(blood)}=A*γ*gₙ⁽²⁾*AWTₙ⁽²⁾, p=3,4.

Performing ranking on all the blood products required by the hospital in a descending order according to z_{n,p}^{(blood)}, and forming the in-hospital blood product supply sequential order table according to a rule of demand tension degree priority.

Further, the Step 5 specifically includes the following steps.

(5.1) Measuring a supply and demand condition of blood products in each hospital,

and building a current dispatching and delivery scheme according to delivery states of the transport tools,

where a blood product inventory in the hospital i is denoted as Iᵢ, and the number of in-transport blood products transported to the hospital i is denoted as Wᵢ;

Wᵢ=Σᵤ₌₁^{U}BU*(I(SUᵤ=i)+Σₖ₌₁^{CU}^{ᵤ}I(RUᵤ^{k}=i))+Σₜ₌₁^{T}BT*(I(STₜ=i)+Σₖ₌₁^{CT}^{ₜ}I(RTₜ^{k}=i))
where U and T are the number of unmanned aerial vehicles and the number of blood delivery cars managed by a blood center respectively, maximum quantities able to be carried by the unmanned aerial vehicles and the blood delivery cars are BU and BT respectively, and I(⋅) is an indicator function.

A set SU={SU₁, . . . , SUᵤ, . . . , SU_{U}} represents a condition of starting a unmanned aerial vehicle, where SUᵤ is valued as 0, i, and −i, which respectively represent that a uth unmanned aerial vehicle is in a standby state in the blood center, on the way to the hospital i, and on the way back to the blood center from the hospital i; CUᵤ is the number of flights scheduled for the uth unmanned aerial vehicle; a set RUᵤ={RUᵤ¹, . . . , RUᵤ^{k}, . . . , RUᵤ^{CU}^{ᵤ}} represents a target hospital where the uth unmanned aerial vehicle is scheduled to fly; RUᵤ^{k}=i represents that the target hospital of a kth flight, scheduled to fly, of the uth unmanned aerial vehicle is the hospital i; a set RU={RU₁, . . . , RUᵤ, . . . , RU_{U}}.

A set ST={ST₁, . . . , STₜ, . . . , ST_{T}} represents a condition of starting a blood delivery car, STₜ is valued as 0, i, and −i, which respectively represent that a tth blood delivery car is in a standby state in the blood center, on the way to the hospital i, and on the way back to the blood center from the hospital i; CTₜ is the number of trips scheduled for the tth blood delivery car; a set RTₜ={RTₜ¹, . . . , RTₜ^{k}, . . . , RTₜ^{CT}^{ₜ}} represents a target hospital where the tth blood delivery car is scheduled to drive; RTₜ^{k}=i represents that the target hospital of a kth trip scheduled for the tth blood delivery car is the hospital i; a set RT={RT₁, . . . , RTₜ, . . . , RT_{T}}.

If a prepared blood volume of the hospital cannot meet a demand blood volume Dᵢ, that is, Iᵢ+Wᵢ<Dᵢ, the hospital is marked to be in a blood-lacking state.

During initial dispatching, Wᵢ=0, all the unmanned aerial vehicles and all the blood delivery cars are in the standby state in the blood center.

The set SU, RU, ST, RT and the in-hospital blood product supply sequential order table of each hospital form a current dispatching and delivery scheme.

(5.2) Gathering all hospitals marked being in the blood-lacking state in a set LH, and obtaining LH={l₁, . . . , lⱼ, . . . , l_{N}_{^{(lack)}}}, where N^{(lack)} is the number of hospitals in the blood-lacking state, and lⱼ represents a jth hospital in the blood-lacking state.

Based on the current dispatching and delivery scheme, calculating an overall future blood product supply tension degree estimated value zⱼ^{(hospital)} of the jth hospital in the blood-lacking state in the set LH as:

$${z}_{j}^{( hospital )} = {\sum }_{n = 1}^{{N}_{{l}_{j}}} {\sum }_{p = 1}^{{R}_{n}} {z}_{n , p}^{( e s t i m a t e d )}$$

where z_{n,p}^{(estimated)} represents a future supply tension degree estimated value of a pth unit of red blood cell blood products of the patient n according to the current dispatching and delivery scheme, and Nₗ_{ⱼ} represents the total number of patients of the jth hospital in the blood-lacking state.

Selecting a hospital with a maximum value in all zⱼ^{(hospital)}, recording as the hospital m, and preferably performing dispatching blood delivery for the hospital.

(5.3) Working out a dispatching scheme with the waiting time of the hospital m being as small as possible based on the unmanned aerial vehicles and the blood delivery cars, including the following steps.

Adopting a cyclic sequence algorithm, taking the minimum waiting time for the blood products of all the patients in the hospital m as a target, working out a next dispatching and delivery scheme based on the current dispatching and delivery scheme through unmanned aerial vehicle priority ranking, comparing the difference between the unmanned aerial vehicles and the blood delivery cars, and adjustment of the indefinite-length route sequences, that is, sending a standby unmanned aerial vehicle to the hospital m, or adding a scheduled flight of the hospital m to a scheduled sequence of a certain unmanned aerial vehicle, or sending a standby blood delivery car to the hospital m, or adding a scheduled trip of the hospital m to a scheduled sequence of a certain blood delivery car.

Firstly, calculating next flight ready time TNᵤ of an unmanned aerial vehicle u of the blood center, performing ascending ranking on TNᵤ, obtaining an unmanned aerial vehicle dispatching ranking table as UAVₗᵢₛₜ={K¹, K², . . . , K^{U}}, and starting dispatching from an unmanned aerial vehicle K¹ with a minimum TNᵤ.

Then, using a dispatching cost function for dispatching strategy evaluation and judgment, and comparing dispatching advantages of two tools by calculating dispatching cost differences of dispatching strategies of the unmanned aerial vehicles and the blood delivery cars.

Sending the unmanned aerial vehicle K¹ with the shortest ready time to load a BU unit of blood products, and obtaining a dispatching cost value as J₁; sending the blood delivery car to load a BT unit of blood products, the BU unit of blood products being used for treating the patient, the remaining being wasted, and obtaining a dispatching cost value as J₂; calculating a dispatching cost difference DeltaJ=J₁−J₂, if DeltaJ<0, dispatching the unmanned aerial vehicle K¹, and otherwise, dispatching the blood delivery car with the shortest ready time.

(5.4) Circularly operating steps (5.1) to (5.3) until supply of the blood products of all the hospitals in the blood-lacking state is met.

Further, in Step 6, if a new trauma patient appears, the number of patients and the blood use demands of the patients in Step 2 are updated, and then steps 3 to 5 are executed; if patient information changes, the blood use demands of the patients in Step 2 are updated, and then Steps 3 to 5 are executed; if the blood product demands of the hospital change due to the changes of the transport route of the patients and the blood type detection status of the patients, the blood product demands of the patients for the hospital are updated, and then Steps 4 to 5 are executed; if the unmanned aerial vehicle or the blood delivery car arrives at a certain hospital, the blood product inventory and the number of in-transport blood products of the hospital are updated, and then Steps 4 to 5 are executed; and if the patients complete blood transfusion at the unmanned aerial vehicle site, the blood product inventory of the hospital affiliated to the unmanned aerial vehicle site and the blood product demands of the patients for the hospital affiliated to the unmanned aerial vehicle site are updated, and then Steps 4 to are executed; and if the patients complete blood transfusion in a certain hospital, the blood product inventory of the hospital and the blood product demands of the patients for the hospital are updated, and then Steps 4 to 5 are executed.

According to a second aspect of the present disclosure, provided is an emergency blood dispatching system based on early prediction and unmanned fast delivery for implementing the method; the apparatus includes two parts: an emergency doctor terminal and a dispatching command platform.

The emergency doctor terminal comprises an information input module and a first communication module; wherein the first communication module sends patient information and receives emergency blood use prediction information of a patient and a recommended scheme of a transport destination of the patient.

The dispatching command platform comprises a second communication module, a demand analysis monitoring module and a dispatching calculation module; wherein the second communication module receives patient information and sends a blood supply demand and dispatching instructions; the demand analysis monitoring module judges an emergency blood use demand condition of the patient and comprehensively evaluates a demand blood volume of a hospital, an in-hospital inventory and an in-transport blood volume condition through an emergency blood use prediction model; and the dispatching calculation module is configured to generate the dispatching instructions of unmanned aerial vehicles and blood delivery cars, and send the instructions through the second communication module.

The present disclosure has the beneficial effects that the emergency blood use prediction model and the unmanned aerial vehicle fast delivery route are introduced, so that the blood use demand of pre-hospital emergency trauma patients is accurately predicted, and the pre-hospital emergency blood transfusion of patients is realized through the unmanned aerial vehicle site, so that the blood supply speed and the treatment quality of patients with traumatic hemorrhage are improved, and the present disclosure is of great value for rescuing trauma patients in remote mountainous areas. When a large-scale traumatic event occurs, the blood consumption increases sharply. The present disclosure evaluates the blood demand of the hospital in real time, and quickly distributes the required blood products from the blood center to the hospital in combination with the unmanned aerial vehicle and the blood delivery car, thus improving the blood supply efficiency of the hospital.

### BRIEF DESCRIPTION OF DRAWINGS

FIG. 1 is a flowchart of an emergency blood dispatching method based on early prediction and unmanned fast delivery provided by an exemplary embodiment;

FIG. 2 is a schematic diagram of an emergency blood scheduling framework based on early prediction and unmanned fast delivery provided by an exemplary embodiment;

FIG. 3 is a structural diagram of an emergency blood dispatching system based on early prediction and unmanned fast delivery provided by an exemplary embodiment;

FIG. 4 is an example of a urban simulation scenario; and

FIG. 5 is an example of a rural simulation scenario.

### DESCRIPTION OF EMBODIMENTS

In order to make the above objects, features and advantages of the present disclosure more obvious and easy to understand, the specific embodiments of the present disclosure will be described in detail with reference to the accompanying drawings.

In the following description, many specific details are set forth in order to fully understand the present disclosure, but the present disclosure can also be implemented in other ways different from those described here, and those skilled in the art can make similar promotion without violating the connotation of the present disclosure, so the present disclosure is not limited by the specific embodiments disclosed below.

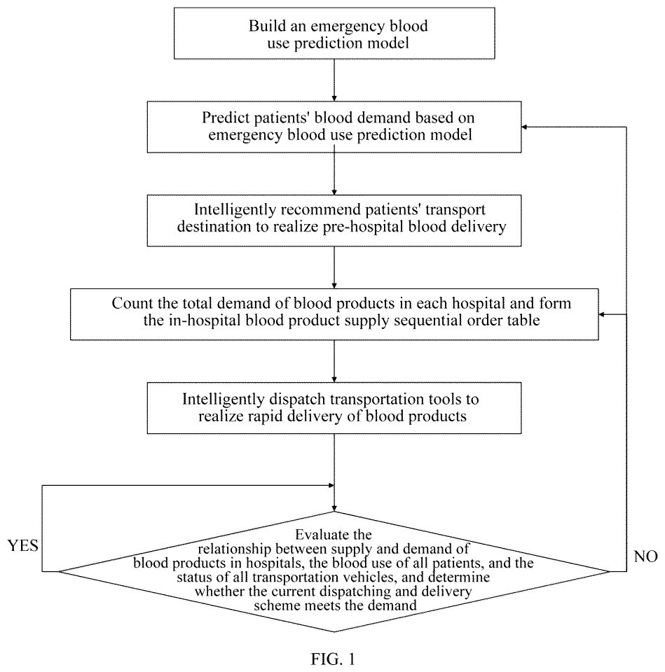

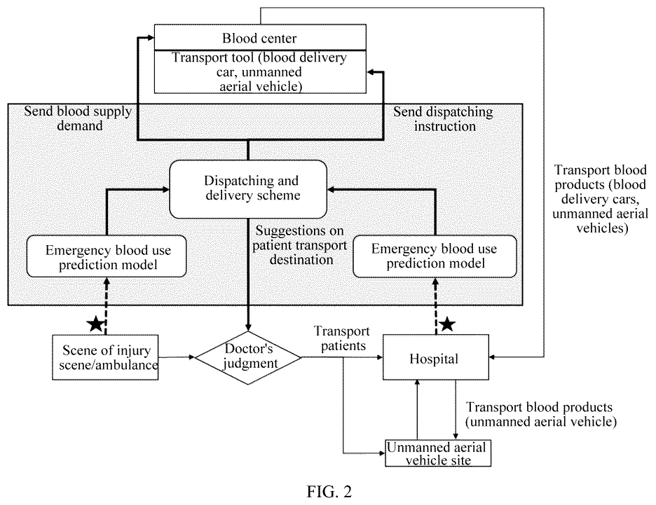

The present disclosure provides an emergency blood dispatching method based on early prediction and unmanned fast delivery, as shown in FIGS. 1 and 2, which includes the following steps:

Step 1, collecting pre-hospital trauma patient samples, and building a staged multi-level emergency blood use prediction model.

Step 2, predicting a blood use demand of a patient based on the emergency blood use prediction model according to trauma patient information.

Step 3, using a two-layer structure weighted composite ratio algorithm to realize intelligent recommendation of a transport destination of the patient and pre-hospital blood delivery through a comparative evaluation with an injury point as a circle center and a weighted triangle comprehensive evaluation according to a location of the patient, and a distance of the patient from surrounding unmanned aerial vehicle sites and surrounding hospitals, to assist an emergency doctor in decision making.

Step 4, counting up total demands of blood products in each hospital, calculating a demand tension degree for all blood products of all patients in each hospital and performing ranking, so as to form an in-hospital blood product supply sequential order table.

Step 5, ranking the priority of the unmanned aerial vehicles, comparing the difference between the unmanned aerial vehicles and blood delivery cars and adjustment of indefinite-length route sequences with a goal of minimizing waiting time constantly and cyclically according to the total demands and a supply tension degree of the blood products in each hospital, an in-hospital inventory, and a number of in-transport blood products based on a circulation sequence algorithm combining the unmanned aerial vehicles and the blood delivery cars, so as to realize intelligent dispatching of transport tools and fast delivery of the blood products.

Step 6, evaluating a supply and demand relationship of the blood products, blood use conditions of all the patients, and states of all the transport tools of each hospital in real time, evaluating whether a current dispatching and delivery scheme meets a demand, and if the scheme does not meet the demand, updating the dispatching and delivery scheme.

The following description further gives some examples of the implementation of the emergency blood dispatching method based on early prediction and unmanned rapid delivery, which meets the requirements of this application.

Step 1, a batch of pre-hospital trauma patients' samples are collected and a staged and multi-level emergency blood use prediction model is constructed, which specifically includes the following steps:

A batch of samples of pre-hospital patients with severe trauma are collected, and the burn patients are excluded, and the sample size is recorded as M; the multi-dimensional pre-hospital and in-hospital information of each selected sample is recorded.

The feature set detected before hospital Xₚᵣₑ^{raw}=[Age, Sex, HR, SBP, DBP, T, SaO₂, flag_{penetrating}, flag_{pelvic}], in which Age, Sex, HR, SBP, DBP, T, SaO₂, flag_{penetrating} and flag_{pelvic} represent age, sex, heart rate, systolic blood pressure, diastolic blood pressure, body temperature, oxygen saturation, penetrating injury and pelvic fracture, respectively.

When the patient is transported to the hospital, more features are collected by blood test and ultrasonic examination in the hospital to form a new feature set Xᵢₙ^{raw}=[HGB, ALB, BE, pH, HCT, flag_{abdominal}], in which HGB, ALB, BE, pH, HCT and flag_{abdominal} respectively represent hemoglobin, albumin, residual alkali, hydrogen ion concentration index, hematocrit and whether there is peritoneal effusion.

The target Y is predicted to be a category k, and a preliminary scheme or an improved scheme may be selected.

In the preliminary scheme, k is valued as 2, and the prediction target Y is whether the 24-hour red blood cell infusion volume is greater than a certain threshold ε, with a value of 1 representing the need for emergency blood, and a value of 0 not being an emergency blood sample. According to the existing research, the threshold ε is set to 4 units. If Y=0, emergency blood use will not be applied, and if Y=1, a blood use for 2 units of O-type red blood cells is applied at a scene of injury immediately.

In the improved scheme, k is valued as 3, and the prediction target Y is 24-hour red blood cell infusion volume belonging to 0 or (0, 4] or (4, +∞), valued as 0, 1 and 2 respectively; if Y=0, blood transfusion is not needed; if Y=1, a blood type is measured after arriving at a hospital, and then a blood use for 2 units of specific blood-type red blood cells is applied; and if Y=2, a blood use for 2 units of O-type red blood cells is applied at the scene of injury immediately, and the blood type is measured after arriving at the hospital, and then a blood use for 2 units of specific blood-type red blood cells is applied.

Firstly, all the features are subjected to mean value vacancy filling and normalization pretreatment to get the pre-hospital feature set Xₚᵣₑ and the new in-hospital feature set Xᵢₙ. Then, a staged multi-level emergency blood use prediction model is constructed based on the feature set after pretreatment by a multi-classification network algorithm. The emergency blood prediction model is expressed as:

$$[ {\hat{y}}_{final}^{0} , … , {\hat{y}}_{final}^{k} , … , {\hat{y}}_{final}^{K - 1} ] = ( 2 - s ) * {f}_{1} ( {X}_{pre} ) + ( s - 1 ) * {f}_{2} ( [ {X}_{pre} , {X}_{i n} ] )$$

$$\hat{Y} = {arg max}_{k} {\hat{y}}_{final}^{k}$$

where s represents a prediction stage, s=1 represents a pre-hospital stage, s=2 represents an in-hospital stage, functions f₁ and f₂ respectively represent a pre-hospital prediction model and an in-hospital prediction model; [Xₚᵣₑ, Xᵢₙ] represents that Xₚᵣₑ, Xᵢₙ are spliced, ŷ_{final}^{k} is a prediction value of the category k output by the staged multi-level emergency blood use prediction model, ŷ_{final}^{k} is valued as [0,1], Ŷ is a predicted blood use category, Ŷ in the preliminary scheme is valued as 0 or 1, and Ŷ in the improved scheme is valued as 0 or 1 or 2.

[ŷₚᵣₑ⁰, . . . , ŷₚᵣₑ^{k}, . . . , ŷₚᵣₑ^{K-1}]=f₁(Xₚᵣₑ)=softmax(W₁·Xₚᵣₑ+b₁)

[ŷᵢₙ⁰, . . . , ŷᵢₙ^{k}, . . . , ŷᵢₙ^{K-1}]=f₂([Xₚᵣₑ, Xᵢₙ])=softmax(W₂·[Xₚᵣₑ, Xᵢₙ]+b₂)

where softmax(⋅) represents a softmax function, W₁, W₂ represent trainable weight parameters, · represents a matrix multiplication; b₁, b₂ represent trainable bias parameters, ŷₚᵣₑ^{k} is a prediction value of the category k output by the pre-hospital prediction model, ŷᵢₙ^{k} is a prediction value of the category k output by the in-hospital prediction model, K is valued as 2 or 3, and ŷₚᵣₑ^{k}, ŷᵢₙ^{k} are valued as [0,1]; when K is valued as 2, representing the preliminary scheme, and when K is valued as 3, representing the improved scheme. Different emergency degrees correspond to different schemes, and the prediction accuracy of patients' blood demand is further improved through the stratification of emergency degrees.

A total loss function Lₜₒₜₐₗ is:

$${L}_{total} = α * {L}_{pre} + ( 1 - α ) * {L}_{i n}$$

$${L}_{pre} = \frac{1}{M} {\sum }_{i = 1}^{M} ( - {\sum }_{j = 0}^{K - 1} I ( {Y}_{i} = j ) ln {\hat{y}}_{pre}^{ij} ) + {λ}_{1} {‖ {W}_{1} ‖}_{2}$$

$${L}_{i n} = \frac{1}{M} {\sum }_{i = 1}^{M} ( - {\sum }_{j = 0}^{K - 1} I ( {Y}_{i} = j ) ln {\hat{y}}_{i n}^{ij} ) + {λ}_{2} {‖ {W}_{2} ‖}_{2}$$

where α is a weight coefficient, Lₚᵣₑ, Lᵢₙ are a pre-hospital prediction model loss function and an in-hospital prediction model loss function respectively, M is a sample size, I(⋅) is an indicator function, Yᵢ is a real category of an ith sample, ŷₚᵣₑ^{ij}, ŷᵢₙ^{ij} are prediction values of categories j of ith samples output by the pre-hospital prediction model and the in-hospital prediction model respectively, λ₁, λ₂ are penalty term coefficients, and ∥⋅∥₂ represents an L2 norm.

Aiming at minimization of Lₜₒₜₐₗ, optimal parameters of the staged multi-level emergency blood use prediction model are obtained by a gradient descent method.

Step 2: the model established in step 1 is used to predict the emergency blood demand of patients, specifically:
for each trauma patient n, the pre-hospital information thereof is input into the staged multi-level emergency blood use prediction model established in step 1, and the model outputs the emergency blood use category Ŷₙ=argmaxₖŷ_{final}^{k} of the patient n; if necessary, blood use for O-type red blood cells is applied. When the patient arrives at the hospital, both pre-hospital information and in-hospital information are input into the staged multi-level emergency blood use prediction model established in step 1, and the emergency blood use prediction result is updated; at the same time, if necessary, after the blood type is determined, an application for red blood cells of a specific blood type is put forward.

In the preliminary scheme, a prediction of 1 represents that emergency blood use is needed, that is, a blood use for 2 units of O-type red blood cell is applied at the scene of injury immediately; a prediction of 0 represents that emergency blood use is not needed.

In the improved scheme, a prediction of 2 represents that a demand for a red blood cell blood product is very emergent, that is, a blood use for 2 units of O-type red blood cells is applied at the scene of injury immediately, the blood type is measured after arriving at the hospital, and then a blood use for 2 units of specific blood-type red blood cells is applied; a prediction of 1 represents that the demand for the red blood cell blood product is in moderate emergency, that is, the blood type is measured after arriving at the hospital, and then a blood use for 2 units of specific blood-type red blood cells is applied; and a prediction of 0 represents that blood transfusion is not needed.

Step 3, a two-layer structure weighted composite ratio algorithm is used to realize intelligent recommendation of a transport destination of the patient and pre-hospital blood delivery through a comparative evaluation with an injury point as a circle center and a weighted triangle comprehensive evaluation according to a location of the patient, and a distance of the patient from surrounding unmanned aerial vehicle sites and surrounding hospitals, to assist an emergency doctor in decision making. The emergency doctor specifies the transport destination for each patient according to the recommended results.

Symbols H and S are the number of hospitals and the number of unmanned aerial vehicle sites in the set area. The hospital location is marked as PH={PH₁, PH₂, . . . , PHᵢ, . . . , PH_{H}}, where PHᵢ represents the location of the ith hospital. The location of the unmanned aerial vehicle site is marked as PS={PS₁, PS₂, . . . , PSⱼ, . . . , PS_{S}}, where PSⱼ represents the location of the jth unmanned aerial vehicle site. The symbol PP stands for the position of a pre-hospital trauma patient. The function MapT(start, end) represents the road traffic time from the starting point start to the ending point end calculated by the map application. There are the following two situations:

I. For the patients who are predicted not to need O-type red blood cells in step 2, the injured point is taken as the center and the time taken to arrive at each hospital is compared, so as to determine which hospital to transfer the patients to for treatment.

The road traffic time THᵢ from the patient's location to the ith hospital location is calculated through the function MapT( ):

THᵢ=MapT(PP, PHᵢ)

The serial number of the hospital with the shortest road traffic time NHI is

selected:

$$NHI = {arg min}_{i} {TH}_{i}$$

It is suggested that the patient be transferred to hospital NHI for treatment, and the patient's blood demand corresponds to hospital NHI.

II. For the patients who are predicted to need O-type red blood cells in step 2, it is judged to transport the patient to a certain unmanned aerial vehicle site to have O-type red blood cell emergency blood transfusion, then transport the patient to a nearby hospital for further treatment, or transport the patient to a certain hospital to have the O-type red blood cell emergency blood transfusion and further treatment; each unmanned aerial vehicle site belongs to a hospital with the shortest unmanned aerial vehicle flight consumption time; in this step, the hospital and unmanned aerial vehicle site with the shortest time to transport patients are obtained by using the comparative evaluation centered on the injury point.

(a) The shortest time to transport the patient from the scene of injury to the hospital by emergency vehicle is calculated, including the following steps.

The road traffic time THᵢ from the patient's location to the i-th hospital location through the function MapT( );

THᵢ=MapT(PP, PHᵢ)

The serial number of the hospital with the shortest road traffic time NHI is selected:

NHI=argminᵢTHᵢ

Therefore, the shortest time to transport patients to the hospital, that is, the time taken by patients to the hospital TNH:

TNH=TH_{NHI}

(b) The shortest time for transporting patients from the scene of injury to the unmanned aerial vehicle site for O-type red blood cell emergency transfusion by emergency vehicle is calculated, including the following steps.

The road traffic time from the patient's location to the location of the jth unmanned aerial vehicle site is calculated through the function MapT( ), and then the time for the patient to obtain O-type red blood cells at the unmanned aerial vehicle site under the condition that the blood inventory of the hospital to which the unmanned aerial vehicle site belongs is sufficient is calculated:

TSⱼ=max(MapT(PP, PSⱼ), Vⱼ)

where max(⋅) is a function of taking the maximum value, and Vⱼ is the flight time from the hospital to which the jth unmanned aerial vehicle site belongs to the jth unmanned aerial vehicle site.

the serial number NSI of the unmanned aerial vehicle site with the smallest TSⱼ is selected:

NSI=argminⱼTSⱼ

Therefore, the shortest time to transport patients to the unmanned aerial vehicle site for emergency blood transfusion TNS:

TNS=TS_{NSI}

(c) The weighted triangle judgment index is calculated to judge the patient's delivery destination, that is, weighted triangle comprehensive evaluation for the hospital NHI and the unmanned aerial vehicle site NSI is performed in this step; the primary factor is that compared with hospital blood transfusion, if the patient can receive emergency blood transfusion as early as possible, the value of blood transfusion at unmanned aerial vehicle site will be greater; the shorter the time it takes for the unmanned aerial vehicle site to transport to the hospital after blood transfusion, the sooner the patient can be further treated after blood transfusion, the better. Therefore, the weighted triangle judgment index C is:

$$C = \frac{T N H}{\sqrt{2 * T N S * ( T N S + 0.5 * T S H )}} - 1$$

where TSH is the road traffic time from the unmanned aerial vehicle site NSI to a hospital Q with shortest consuming time to the site.

The output of this step is DEST, which contains the type and specific location information of the patient's transport destination.

If the index C is greater than 0, DEST=(“station”, NSI, “hospital”, Q) is output, and it is suggested to transport the patient to the unmanned aerial vehicle site NSI for O-type red blood cell emergency blood transfusion, then transport the patient to the hospital Q for further treatment, supply the blood use demand of the patient at the unmanned aerial vehicle site NSI by the hospital affiliated to the unmanned aerial vehicle site, and supply the blood use demand for further treatment by the hospital Q; and otherwise, DEST=(“hospital”, NHI) is output, and it is suggested to transport the patient to the hospital NHI for O-type red blood cell emergency blood transfusion and further treatment, and the blood use demand of the patient corresponds to the hospital NHI.

Step 4, the total demand for blood products in each hospital at time t is counted, a demand tension degree for all blood products of all patients in each hospital is calculated and ranking performed, so as to form an in-hospital blood product supply sequential order table.

The number of all patients in a hospital i at time t is recorded as Nᵢ, including patients transported to the hospital from the scene of injury or the unmanned aerial vehicle site, and patients having emergency blood transfusion at the unmanned aerial vehicle site managed by the hospital.

(4.1) The total demand for blood products in each hospital is counted; for the patient n, the staged multi-level emergency blood use prediction model is adopted to predict a category Ŷₙ, and calculation is performed to obtain the number Rₙ of red blood cell blood product demands of the hospital for the treatment of the patient n through Ŷₙ, a patient treatment route, and a patient blood type determination status.

In the preliminary scheme, if Ŷₙ=0, Rₙ=0; if Ŷₙ=1, whether the emergency blood product of the patient n is supplied by the hospital is judged; if O-type red blood cell emergency blood transfusion is performed at the hospital or the unmanned aerial vehicle site managed by the hospital, Rₙ=2, and if the hospital is not required to prepare the emergency blood product of the patient n, Rₙ=0.

In the improved scheme, if Ŷₙ=0, Rₙ=0; if Ŷₙ=1, whether a blood type of the patient n has been determined at time t is judged; if the blood type is not determined, Rₙ=0, if the blood type has been determined, Rₙ=2, if Ŷₙ=2, whether O-type red blood cells for emergency blood transfusion of the patient n are supplied by the hospital is judged, whether the specific blood-type red blood cells for further treatment are supplied by the hospital, and whether the blood type of the patient n has been determined at time t are judged; if all the red blood cells of the patient n are supplied by the hospital and the blood typed is not determined, Rₙ=2, if all the red blood cells of the patient n are supplied by the hospital and the blood type has been determined, Rₙ=4; if for the patient n, only the O-type red blood cells are supplied by the hospital, Rₙ=2; if for the patient n, only the specific blood-type red blood cells are supplied by the hospital and the blood type is not determined, Rₙ=0; and if for the patient n, only the specific blood-type red blood cells are supplied by the hospital and the blood type has been determined, Rₙ=2.

The blood use demands of all the patients in the hospital are gathered, and the total blood product demands at time t are evaluated; the total demand of the blood products of the hospital i at time t is Dᵢ=Σₙ₌₁^{N}^{ᵢ}Rₙ.

(4.2) The demand tension degree of all the blood products of all the patients in each hospital is calculated and ranking is performed, so as to form the in-hospital blood product supply sequential order table; for the patient n in the hospital i, according to the prediction result of emergency blood demand obtained in step 2, a blood use tension degree zₙ^{(patient)} of the patient n in the hospital i is calculated in combination with a duration of the patient n waiting for the blood product (with a unit of minute), and a demand tension degree z_{n,p}^{(blood)}, p=1, . . . , Pₙ of all red blood cells of the patient n in the hospital i is calculated according to zₙ^{patient)}, where Pₙ is a total demand for the red blood cells of the patient n.

In the preliminary scheme, if Ŷₙ=0, zₙ^{(patient)}=0; if Ŷₙ=1, zₙ^{(patient)}=gₙ*AWTₙ, where gₙ represents whether the emergency blood product of the patient n is supplied by the hospital; if the blood product is supplied by the hospital, gₙ=1, otherwise, gₙ=0, AWTₙ represents the time spent by the patient n in waiting for the emergency blood product at time t. If Ŷₙ=0, no z_{n,p}^{(blood)}; and if Ŷₙ=1, the demand tension degree of the blood product is z_{n,p}^{(blood)}=zₙ^{(patient)}, p=1,2, that is, the demand tension degree of the blood product in the first unit and the second unit is the same, which is equal to zₙ^{(patient)}.

In the improved scheme, if Ŷₙ=0 , zₙ^{(patient)}=0; if Ŷₙ=1, zₙ^{(patient)}=gₙ*AWTₙ, where gₙ represents whether the emergency blood product of the patient n is supplied by the hospital and whether the blood type has been determined; if the emergency blood product is supplied by the hospital and the blood type has been determined, gₙ=1, and otherwise gₙ=0, AWTₙ represents the time spent by the patient n in waiting for the emergency blood product at time t. If Ŷₙ=2, zₙ^{(patient)}=A*(gₙ⁽¹⁾*AWTₙ⁽¹⁾+γ*gₙ⁽²⁾*AWTₙ⁽²⁾), where A is a ratio coefficient of importance of blood transfusion for very emergent patients to importance of blood transfusion for moderate emergent patients, A>1 , gₙ⁽¹⁾, gₙ⁽²⁾ represents whether the O-type red blood cell blood product for first emergency treatment of the patient n is supplied by the hospital, whether the specific blood-type red blood cells for further treatment are supplied by the hospital and whether the blood type has been determined respectively; if the O-type red blood cell blood product for first emergency treatment is supplied by the hospital, gₙ⁽¹⁾=1, otherwise gₙ⁽¹⁾=0; and if the specific blood-type red blood cells for further treatment are supplied by the hospital and the blood type has been determined, gₙ⁽²⁾=1, otherwise gₙ⁽²⁾=0, where AWTₙ⁽¹⁾, AWTₙ⁽²⁾ represents the time spent by the patient n in waiting for O-type red blood cells required for first emergency treatment at time t and time spent by the patient n in waiting for the specific blood-type red blood cells required for further treatment at time t respectively, γ is a value discount factor of the specific blood-type red blood cells required for further treatment, and γ∈[0,1); if Ŷₙ=0, there is no z_{n,p}^{(blood)}; if Ŷₙ=1, the demand tension degree for the blood product is z_{n,p}^{(blood)}=zₙ^{(patient)}, p=1,2; if Ŷₙ=2, the demand tension degree for the blood product is z_{n,p}^{(blood)}=A*gₙ⁽¹⁾*AWTₙ⁽¹⁾, p=1,2, but z_{n,p}^{(blood)}=A*γ*gₙ⁽²⁾*AWTₙ⁽²⁾, p=3,4.

All the blood products required by the hospital are ranked in a descending order according to z_{n,p}^{(blood)}, and the in-hospital blood product supply sequential order table is formed according to a rule of demand tension degree priority. If two blood products with the same demand tension are encountered, they should be sorted in descending order according to their patients, and then sorted in a random way.

Step 5, priority ranking of the unmanned aerial vehicles, difference comparison between the unmanned aerial vehicles and blood delivery cars and adjustment of indefinite-length route sequences with a goal of minimizing waiting time are constantly and cyclically performed according to the total demands and a supply tension degree of the blood products in each hospital, an in-hospital inventory, and a number of in-transport blood products based on a circulation sequence algorithm combining the unmanned aerial vehicles and the blood delivery cars, so as to realize intelligent dispatching of transport tools and fast delivery of the blood products. Specifically:

The supply and demand of blood products and the supply tension of blood products in each hospital are evaluated by synthesizing the conditions of all patients to be sent to the hospital or the drone site managed by the hospital. By comparing the blood tension degree in all hospitals, we can determine how to dispatch the unmanned aerial vehicle or blood delivery car for rapid dispatching of blood products.

(5.1) A supply and demand condition of blood products in each hospital is measured, and a current dispatching and delivery scheme is built according to delivery states of the transport tools.

A blood product inventory in the hospital i is recorded as Iᵢ, and the number of in-transport blood products transported to the hospital i is recorded as Wᵢ;

$${W}_{i} = {\sum }_{u = 1}^{U} B U * ( I ( {SU}_{u} = i ) + {\sum }_{k = 1}^{C {U}_{u}} I ( {RU}_{u}^{k} = i ) ) + {\sum }_{t = 1}^{T} B T * ( I ( {ST}_{t} = i ) + {\sum }_{k = 1}^{C {T}_{t}} I ( {RT}_{t}^{k} = i ) )$$

where U and T are the number of unmanned aerial vehicles and the number of blood delivery cars managed by a blood center respectively, maximum quantities able to be carried by the unmanned aerial vehicles and the blood delivery cars are BU and BT respectively, and I(⋅) is an indicator function; a set SU={SU₁, . . . , SUᵤ, . . . , SU_{U}} represents a condition of starting a unmanned aerial vehicle, where SU is valued as 0, i, and −i, which respectively represent that a uth unmanned aerial vehicle is in a standby state in the blood center, on the way to the hospital i and on the way back to the blood center from the hospital i. CUᵤ is the number of flights scheduled for the uth unmanned aerial vehicle; a set RU={RUᵤ¹, . . . , RUᵤ^{k}, . . . , RUᵤ^{CU}^{ᵤ}} represents a target hospital where the uth unmanned aerial vehicle is scheduled to fly. RUᵤ^{k}=i represents that the target hospital of a kth flight, scheduled to fly, of the uth unmanned aerial vehicle is the hospital i. A set RU={RU₁, . . . , RUᵤ, . . . , RU_{U}}, and a set ST={ST₁, . . . , STₜ, . . . , ST_{T}}, representing a condition of starting a blood delivery car. STₜ is valued as 0, i, and −I, which respectively represent that a tth blood delivery vehicle is in a standby state in the blood center, on the way to the hospital i, and on the way back to the blood center from the hospital i. CTₜ is the number of trips scheduled for the tth blood delivery car; a set RTₜ={RTₜ¹, . . . , RTₜ^{k}, . . . , RTₜ^{CT}^{ₜ}} represents a target hospital where the tth blood delivery car is scheduled to drive, RTₜ^{k}=i represents that the target hospital of a kth trip scheduled for the tth blood delivery car is the hospital i; and a set RT={RT₁, . . . , RTₜ, . . . , RTₜ}.

If a prepared blood volume of the hospital cannot meet a demand blood volume Dᵢ, that is, Iᵢ+Wᵢ<Dᵢ, the hospital is marked to be in a blood-lacking state.

During initial dispatching, Wᵢ=0, all the unmanned aerial vehicles and all the blood delivery cars are in the standby state in the blood center.

The set SU, RU, ST, RT and the in-hospital blood product supply sequential order table of each hospital form a current dispatching and delivery scheme.

(5.2) The hospitals with insufficient supply of blood products, that is, the hospitals in blood-lacking state, are gathered, the overall future supply tension of blood products in these hospitals is evaluated, and the hospitals that are preferably scheduled for dispatching are selected.

All hospitals marked being in the blood-lacking state are gathered in a set LH to obtain LH={l₁, . . . , lⱼ, . . . , l_{N}_{^{(lack)}}}, where N^{(lack)} is the number of hospitals in the blood-lacking state, and represents a jth hospital in the blood-lacking state;

Based on the current dispatching and delivery scheme, an overall future blood product supply tension degree estimated value zⱼ^{(hospital)} of the jth hospital in the blood-lacking state in the set LH is calculated as:

$${z}_{j}^{( hospital )} = {\sum }_{n = 1}^{{N}_{{l}_{j}}} {\sum }_{p = 1}^{{R}_{n}} {z}_{n , p}^{( e s t i m a t e d )}$$

where z_{n,p}^{(estimated)} represents a future supply tension degree estimated value of a pth unit of red blood cell blood products of the patient n according to the current dispatching and delivery scheme, and Nₗ_{ⱼ} represents the total number of patients of the jth hospital in the blood-lacking state.

In the preliminary scheme, if Ŷₙ=0Ŷₙ=0, there is no z_{n,p}^{(estimated)}; if Ŷₙ=1, the estimated blood product supply tension degree in the future z_{n,p}^{(estimated)}=gₙ*EWTₙ, p=1,2, where EWTₙ indicates the estimated time for the patient n to wait for emergency blood products according to the current dispatching and delivery scheme. If the current dispatching and delivery scheme cannot meet the demand for blood products required by patient n, EWTₙ will be set to a larger fixed value, for example EWTₙ=24*60=1440 minutes.

In the improved scheme, if Ŷₙ=0, there is no z_{n,p}^{(estimated)}; if Ŷₙ=1, the estimated value of the supply tension degree of the blood products in the future z_{n,p}^{(estimated)}=gₙ*EWTₙ, p=1,2; if Ŷₙ=2, z_{n,p}^{(estimated)}=A*gₙ⁽¹⁾*EWTₙ⁽¹⁾, p=1,2, and z_{n,p}^{(estimated)}=A*γ*gₙ⁽²⁾*EWTₙ⁽²⁾, p=3,4, where EWTₙ⁽¹⁾, EWTₙ⁽²⁾ respectively represent the estimated time for the patient n to wait for the O-type red blood cells in the first step of emergency treatment and the estimated time for waiting for the red blood cells of a specific blood type for further treatment according to the current dispatching and delivery scheme; if the current dispatching and delivery scheme cannot meet the demand for blood products of the first, second or third, fourth units required by the patient n, EWTₙ⁽¹⁾ or EWTₙ⁽²⁾ will be set to a larger fixed value, for example EWTₙ⁽¹⁾=1440 minutes or EWTₙ⁽²⁾=1440 minutes.

The hospital with a maximum value in all zⱼ^{(hospital)} is selected, and recorded as the hospital m, and dispatching blood delivery is preferably performed for the hospital, i.e., the next step is performed.

(5.3) A dispatching scheme with the waiting time of the hospital m being as small as possible is worked out based on the unmanned aerial vehicles and the blood delivery cars.

A cyclic sequence algorithm is adopted, the minimum waiting time for the blood products of all the patients in the hospital m is taken as a target, a next dispatching and delivery scheme is worked out based on the current dispatching and delivery scheme through unmanned aerial vehicle priority ranking, comparing the difference between the unmanned aerial vehicles and the blood delivery cars, and adjustment of the indefinite-length route sequences, that is, sending a standby unmanned aerial vehicle to the hospital m, or adding a scheduled flight of the hospital m to a scheduled sequence of a certain unmanned aerial vehicle, or sending a standby blood delivery car to the hospital m, or adding a scheduled trip of the hospital m to a scheduled sequence of a certain blood delivery car;

Firstly, the next flight ready time TNᵤ of an unmanned aerial vehicle u of the blood center and its ranking are calculated. For unmanned aerial vehicle in standby state, TNᵤ=0; for unmanned aerial vehicles that are on the road and have no scheduled flight, TNᵤ=TRᵤ, where TRᵤ is the time required for the unmanned aerial vehicle u to end the current flight; for unmanned aerial vehicles in other states, then:

TNᵤ=T [symbol] +Σₖ₌₁^{CU}^{ᵤ}2*TUC

[symbol]

where [symbol] is the number of flights scheduled for the [symbol] th unmanned aerial vehicle, RUᵤ^{k} is the target hospital for the kth scheduled flight of the unmanned aerial vehicle [symbol] , and TUC [symbol] indicates the flight time of the unmanned aerial vehicle from the blood center to the hospital [symbol] .

Ascending ranking is performed on [symbol] , and an unmanned aerial vehicle dispatching ranking table is UAVₗᵢₛₜ={K¹, K², . . . , K^{U}}, and dispatching is started from an unmanned aerial vehicle, i.e., K¹, with a minimum [symbol] .

Then, a dispatching cost function is used for dispatching strategy evaluation and judgment, and dispatching advantages of two tools are compared by calculating dispatching cost differences of dispatching strategies of the unmanned aerial vehicles and the blood delivery cars.

In the preliminary scheme, the dispatching cost function is:

J=Σₙ₌₁^{N}^{ₘ}I(Ŷₙ=1)*gₙ*FTₙ+β*C^{Blood}

where I(⋅) is an indicator function, Nₘ is the number of patients in hospital m, FTₙ represents the estimated time for the patient n to wait for emergency blood products according to the next dispatching and delivery scheme. If the dispatching and delivery scheme cannot meet the demand for blood products needed by the patient n, FTₙ is set c to be a larger fixed value, for example, let FTₙ equal to 1440 minutes. β represents the penalty coefficient of blood product waste, which is determined by clinical experience and blood product inventory in the blood center. C^{Blood} represents the amount of blood products wasted due to oversupply.

In the improved scheme, the dispatching cost function is:

J=Σₙ₌₁^{N}^{ₘ}(I(Ŷₙ=1)*gₙ*FTₙ+I(Ŷₙ=2)*A*(gₙ⁽¹⁾*FTₙ⁽¹⁾+γ*gₙ⁽²⁾*FTₙ⁽²⁾))+β*C^{Blood}

where I(⋅) is an indicator function, FTₙ⁽¹⁾, FTₙ⁽²⁾ respectively represent the estimated time for the patient n to wait for the O-type red blood cells in the first step of emergency treatment and the estimated time for waiting for the red blood cells of a specific blood type for further treatment according to the next dispatching and delivery scheme; if the dispatching and delivery scheme cannot meet the needs of O-type red blood cells or red blood cells of a specific blood type required by the patient n, FTₙ⁽¹⁾ or FTₙ⁽²⁾ is set to a larger fixed value, for example FTₙ⁽¹⁾=1440 minutes or FTₙ⁽²⁾=1440 minutes. β represents the penalty coefficient of blood product waste, which is determined by clinical experience and blood product inventory in the blood center. C^{Blood} represents the amount of blood products wasted due to oversupply.

In order to be comparable, Scheme 1 is to dispatch the unmanned aerial vehicle K¹ with the shortest ready time (loaded with BU units of blood products), and the dispatching cost is J₁. Scheme 2 is to send a blood delivery car (carrying BT units of blood products, with BU units of blood products used to treat patients, and the rest wasted), and the dispatching cost is J₂. The dispatching cost difference DeltaJ=J₁−J₂ is calculated. If DeltaJ<0, the unmanned aerial vehicle K¹ is used and the use mode thereof is judged according to its state; if TN_{K}_{¹} is equal to 0, the unmanned aerial vehicle K¹ is immediately dispatched to transport blood products to the hospital m; if TN_{K}_{¹} is greater than 0, a scheduled flight of the hospital m will be added to the scheduling list RU_{K}_{¹}, and at the same time, the number of blood products Wₘ in transit, the ready time TN_{K}_{¹} and the scheduling list UAVₗᵢₛₜ of the unmanned aerial vehicle will be updated; if DeltaJ≥0, the blood delivery car with the shortest ready time is dispatched; the specific operation is consistent with the above operation using the unmanned aerial vehicle.

(5.4) Steps (5.1) to (5.3) are circularly operated until supply of the blood products of all the hospitals in the blood-lacking state is met. Specifically: the supply and demand of blood products in each hospital is calculated, that is, step (5.1) is executed again. If there are still hospitals with insufficient supply, step (5.2) is executed again, and the hospitals with insufficient blood supply that need further dispatching are selected; step (5.3) is executed, and the unmanned aerial vehicle or blood delivery car is dispatched to deliver blood products. The above steps are repeated until the supply of blood products in all hospitals with insufficient blood supply have been satisfied before exiting.

Step 6, a supply and demand relationship of the blood products, blood use conditions of all the patients, and states of all the transport tools of each hospital are evaluated in real time, whether a current dispatching and delivery scheme meets a demand is evaluated when any variable in step 2 to step 5 changes, and if the current scheme does not meet the demand, the dispatching and delivery scheme is updated. If there are new trauma patients, the number of patients and the blood demand of

patients in step 2 are updated, and then steps 3 to 5 are executed; if the patient information changes, the patient's blood demand in step 2 is updated, and then steps 3 to 5 are executed; if the patient's transport route, the patient's blood type test status and other changes lead to changes in the demand for blood products in the hospital, the patient's demand for blood products in the hospital is updated, and then steps 4 and 5 are executed; if the unmanned aerial vehicle or blood delivery car arrives at a hospital, the blood product inventory and the number of blood products in transit in the hospital are updated, and then steps 4 and 5 are executed; if the patient completes blood transfusion at a unmanned aerial vehicle site, the inventory of blood products in the hospital to which the unmanned aerial vehicle site belongs is updated, and the patient's demand for blood products in the hospital to which the unmanned aerial vehicle site belongs is updated, and then steps 4 and 5 are executed; if the patient completes blood transfusion in a hospital, the inventory of blood products in the hospital is updated, the patient's demand for blood products in the hospital is updated, and then steps 4 and 5 are executed.

Corresponding to the aforementioned embodiment of the emergency blood dispatching method based on early prediction and unmanned fast delivery, the present disclosure also provides an embodiment of an emergency blood dispatching system based on early prediction and unmanned fast delivery.

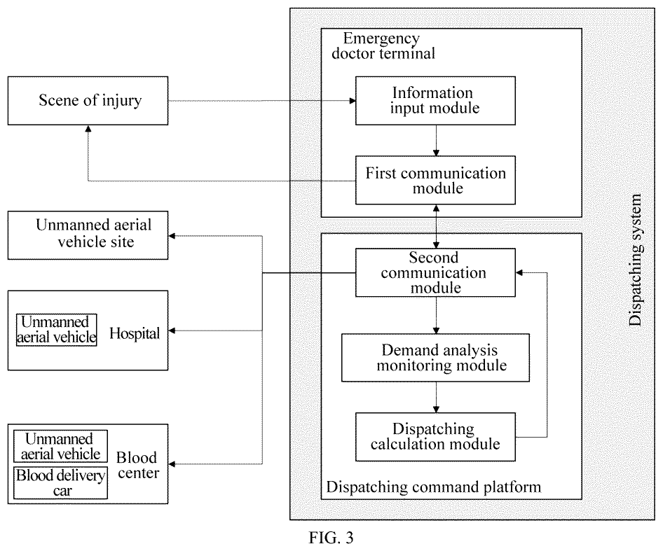

As shown in FIG. 3, an emergency blood dispatching system based on early prediction and unmanned fast delivery provided by an embodiment of the present disclosure includes an emergency doctor terminal and a dispatching command platform.

The emergency doctor terminal includes an information input module and a first communication module. The first communication module sends patient information and receives emergency blood use prediction information of a patient and a recommended scheme of a transport destination of the patient.

The dispatching command platform incudes a second communication module, a demand analysis monitoring module and a dispatching calculation module. The specific functions are as follows: the second communication module receives patient information and sends a blood supply demand and dispatching instructions; the demand analysis monitoring module judges an emergency blood use demand condition of the patient and comprehensively evaluates a demand blood volume of a hospital, an in-hospital inventory and an in-transport blood volume condition through an emergency blood use prediction model; and the dispatching calculation module is configured to generate the dispatching instructions of unmanned aerial vehicles and blood delivery cars, and send the instructions through the second communication module.

In the following examples, application scenarios and simulation results of the present disclosure will be explained.

(1) A simulation experiment in the present disclosure shows that the waiting time is reduced and the emergency blood supply efficiency is improved.

The dispatching method and system of the present disclosure are tested in two simulation experiments, which respectively simulate the urban characteristic scenario and the rural characteristic scenario, and are realized based on AnyLogic software (free version).

### a. Simulation Experiment of Urban Characteristics

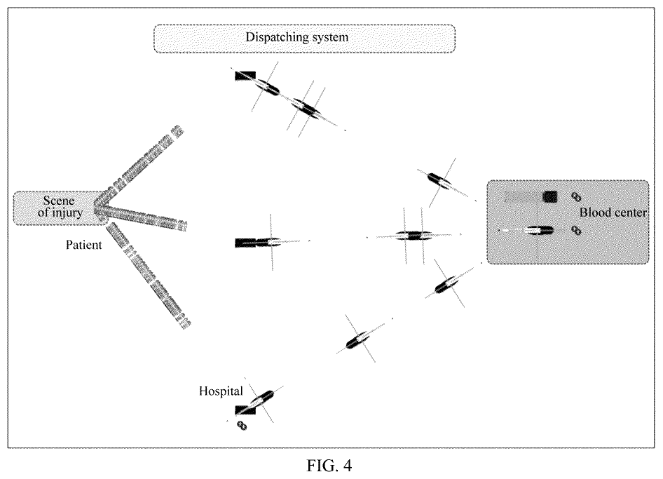

This scenario includes a blood center, hospitals and a scene of injury. Among them, the blood center has a number of blood delivery cars and unmanned aerial vehicles, and there will be a number of severely traumatized patients at the scene of injury and some patients need emergency blood transfusion. The urban simulation scenario is shown in FIG. 4.

As a result, when there are 300 trauma patients at the scene of injury, the average waiting time for blood transfusion of the traditional strategy is 30.52 minutes, while the average waiting time for blood transfusion required by the dispatching system of the present disclosure is 17.55 minutes.

### b. Simulation Experiment of Rural Characteristics

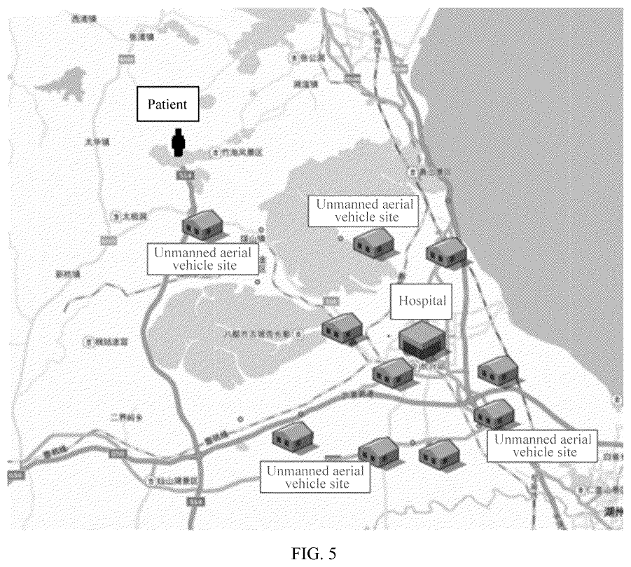

The scenario includes hospitals, unmanned aerial vehicle sites, and a scene of injury. Among them, the hospitals have a number of unmanned aerial vehicles, and there will be a number of seriously injured patients at the scene of injury and some patients need emergency blood transfusion. The rural simulation scenario is shown in FIG. 5.

As a result, the average waiting time for blood transfusion in the traditional strategy is 89.37 minutes, while the average waiting time for blood transfusion in the dispatching system of the present disclosure is 42.05 minutes.

(2) The dispatching method and system of the present disclosure have been put into real operation and proved to be feasible in 11 cases. It takes 21-132 minutes from the reception to the arrival of the patient in the hospital, and it takes only 5 minutes for the unmanned aerial vehicle to fly from the blood center to the hospital, which can reach a ready state before the patient arrives.

The above is only the preferred embodiment of the present disclosure, and although the present disclosure has been disclosed in the above with preferred embodiments, it is not intended to limit the present disclosure. A person skilled in the art can make many possible changes and modifications to the technical solution of the present disclosure by using the methods and technical contents disclosed above, or modify it into equivalent embodiments with equivalent changes without departing from the scope of the technical scheme of the present disclosure. Therefore, any simple modification, equivalent change and modification of the above embodiment according to the technical essence of the present disclosure that does not depart from the content of the technical scheme of the present disclosure still falls within the scope of protection of the technical scheme of the present disclosure.

What is claimed is:

1. A1. An emergency blood dispatching method based on early prediction and unmanned fast delivery, comprising:
step 1, collecting pre-hospital trauma patient samples, and building a staged multi-level emergency blood use prediction model, comprising:
collecting the pre-hospital trauma patient samples, and recording pre-hospital and in-hospital multidimensional information; a prediction target Y being a k category, and selecting a preliminary scheme or an improved scheme according to an emergency degree;
wherein in the preliminary scheme, k is valued as 2, and the prediction target Y is whether 24-hour red blood cell infusion volume belongs to [0, 4] or (4, +∞), valued as 0 or 1, respectively; if Y=0, emergency blood use is not applied; and if Y=1, a blood use for 2 units of O-type red blood cells is immediately applied at injury scene;
wherein in the improved scheme, k is valued as 3, and the prediction target Y is whether the 24-hour red blood cell infusion volume belongs to 0, (0, 4] or (4, +∞), valued as 0, 1 or 2, respectively; if Y=0, blood transfusion is not needed; if Y=1, a blood type is measured after arriving at a hospital and a blood use for 2 units of specific blood-type red blood cells is applied; and if Y=2, the blood use for 2 units of O-type red blood cells is immediately applied at the injury scene, and the blood type is measured after arriving at the hospital, and the blood use for 2 units of specific blood-type red blood cells is applied;
the staged multi-level emergency blood use prediction model is represented as:
$$[ {\hat{y}}_{final}^{0} , … , {\hat{y}}_{final}^{k} , … , {\hat{y}}_{final}^{K - 1} ] = ( 2 - s ) * {f}_{1} ( {X}_{p r e} ) + ( s - 1 ) * {f}_{2} ( [ {X}_{pre} , {X}_{i n} ] )$$
$$\hat{y} = {arg max}_{k} {\hat{y}}_{final}^{k}$$
where s represents a prediction stage, s=1 represents a pre-hospital stage, s=2 represents an in-hospital stage, functions f₁ and f₂ respectively represent a pre-hospital prediction model and an in-hospital prediction model, Xₚᵣₑ, Xᵢₙ respectively represent a pre-hospital feature set and an in-hospital newly-added feature set after mean value vacancy filling and normalization pretreatment; [Xₚᵣₑ, Xᵢₙ] represents that Xₚᵣₑ, Xᵢₙ are spliced, ŷ_{final}^{k} is a prediction value of the category k output by the staged multi-level emergency blood use prediction model, ŷ_{final}^{k} is valued as [0,1], Ŷ is a predicted blood use category, Ŷ in the preliminary scheme is valued as 0 or 1, and Ŷ in the improved scheme is valued as 0, 1 or 2;
wherein in the staged multi-level emergency blood use prediction model:
[ŷₚᵣₑ⁰, . . . , ŷₚᵣₑ^{k}, . . . , ŷₚᵣₑ^{K-1}]=f₁(Xₚᵣₑ)=softmax(W₁·Xₚᵣₑ+b₁)
[ŷᵢₙ⁰, . . . , ŷᵢₙ^{k}, . . . , ŷᵢₙ^{K-1}]=f₂([Xₚᵣₑ, Xᵢₙ])=softmax(W₂·[Xₚᵣₑ, Xᵢₙ]+b₂)
where softmax(⋅) represents a softmax function, W₁, W₂ represent trainable weight parameters, · represents a matrix multiplication; b₁, b₂ represent trainable bias parameters, ŷₚᵣₑ^{k} prediction value of the category k output by the pre-hospital prediction model, ŷᵢₙ^{k} is a prediction value of the category k output by the in-hospital prediction model, k is valued as 2 or 3, and ŷₚᵣₑ^{k}, ŷᵢₙ^{k} are valued as [0,1]; when k is valued as 2, representing the preliminary scheme, and when k is valued as 3, representing the improved scheme; and
wherein a total loss function Lₜₒₜₐₗ is:
$${L}_{total} = α * {L}_{pre} + ( 1 - α ) * {L}_{i n}$$
$${L}_{pre} = \frac{1}{M} {\sum }_{i = 1}^{M} ( - {\sum }_{j = 0}^{K - 1} I ( {Y}_{i} = j ) ln {\hat{y}}_{pre}^{i j} ) + {λ}_{1} {‖ {W}_{1} ‖}_{2}$$
$${L}_{i n} = \frac{1}{M} {\sum }_{i = 1}^{M} ( - {\sum }_{j = 0}^{K - 1} I ( {Y}_{i} = j ) ln {\hat{y}}_{i n}^{i j} ) + {λ}_{2} {‖ {W}_{2} ‖}_{2}$$
where α is a weight coefficient, Lₚᵣₑ, Lᵢₙ are a pre-hospital prediction model loss function and an in-hospital prediction model loss function, respectively, M is a sample size, I(⋅) is an indicator function, Yᵢ is a real category of an ith sample, ŷₚᵣₑ^{ij}, ŷᵢₙ^{ij} are prediction values of categories j of ith samples output by the pre-hospital prediction model and the in-hospital prediction model, respectively, λ₁, λ₂ are penalty term coefficients, and ∥⋅∥₂ represents an L2 norm; and
aiming at minimization of Lₜₒₜₐₗ to obtain optimal parameters of the staged multi-level emergency blood use prediction model by a gradient descent method;
step 2, predicting a blood use demand of a patient based on the staged multi-level emergency blood use prediction model according to trauma patient information;
step 3, using a two-layer structure weighted composite ratio algorithm to realize intelligent recommendation of a transport destination of the patient and pre-hospital blood delivery through a comparative evaluation with an injury point as a circle center and a weighted triangle comprehensive evaluation according to a location of the patient, and a distance of the patient from surrounding unmanned aerial vehicle sites and surrounding hospitals, to assist an emergency doctor to make a decision;
step 4, counting a total demand for blood products in each hospital, and calculating and ranking a demand tension degree for all blood products of all patients in each hospital, so as to form an in-hospital blood product supply sequential order table;
step 5, ranking priority of the unmanned aerial vehicles, comparing a difference between the unmanned aerial vehicles and blood delivery cars and adjusting indefinite-length route sequences with a goal of minimizing waiting time constantly and cyclically according to the total demand for the blood products, a supply tension degree of the blood products, an in-hospital inventory, and a number of in-transport blood products in each hospital based on a circulation sequence algorithm combining the unmanned aerial vehicles and the blood delivery cars, so as to realize intelligent dispatching of transport tools and fast delivery of the blood products; and
step 6, evaluating a supply and demand relationship of blood products, blood use conditions of all the patients, and states of all transport tools of each hospital in real time, evaluating whether a current dispatching and delivery scheme satisfy a demand, and when the scheme does not satisfy the demand, updating the dispatching and delivery scheme.

2. The emergency blood dispatching method based on early prediction and unmanned fast delivery according to claim 1, wherein the step 2 comprises:
inputting, for each trauma patient, the pre-hospital information of the patient into the staged multi-level emergency blood use prediction model built in step 1, and outputting an emergency blood use category of the patient; when the patient arrives at the hospital, inputting both the pre-hospital information and in-hospital information of the patient into the staged multi-level emergency blood use prediction model built in step 1 to update an emergency blood use prediction result;
wherein in the preliminary scheme, a prediction of 1 represents that emergency blood use is needed, the blood use for 2 units of O-type red blood cell is immediately applied at the injury scene, and a prediction of 0 represents that emergency blood use is not needed; and
wherein in the improved scheme, a prediction of 2 represents that a demand for a red blood cell blood product is very emergent, a blood use for 2 units of O-type red blood cells is immediately applied at the injury scene, the blood type is measured after arriving at the hospital, and the blood use for 2 units of specific blood-type red blood cells is applied; a prediction of 1 represents that the demand for the red blood cell blood product is moderately emergent, the blood type is measured after arriving at the hospital, and the blood use for 2 units of specific blood-type red blood cells is applied; and a prediction of 0 represents that blood transfusion is not needed.

3. The emergency blood dispatching method based on early prediction and unmanned fast delivery according to claim 2, wherein the step 3 comprises the following two conditions:
condition 1: for a patient predicted not to need the O-type red blood cells in step 2, road traffic time arriving at each hospital is compared by taking the injury point as the circle center, suggesting transporting the patient predicted not to need the O-type red blood cells to a hospital NHI with a shortest road traffic time for treatment, and a blood use demand of the patient corresponding to the hospital NHI;
condition 2: for the patient predicted to need the O-type red blood cells in step 2, determining to transport the patient predicted to need the O-type red blood cells to a certain unmanned aerial vehicle site for O-type red blood cell emergency blood transfusion, and then transport the patient predicted to need the O-type red blood cells to a nearby hospital for further treatment, or to transport the patient predicted to need the O-type red blood cells to a certain hospital for the O-type red blood cell emergency blood transfusion and further treatment; and each unmanned aerial vehicle site belonging to a hospital with a shortest unmanned aerial vehicle flight consumption time; comprising:
(a) a shortest road traffic time TNH for transporting the patient from the injury scene to the hospital by an emergency vehicle is calculated, and a hospital serial number NHI corresponding to the TNH is recorded;
(b) a shortest time TNS for transporting the patient from the injury scene to the unmanned aerial vehicle site by the emergency vehicle for the O-type red blood cell emergency blood transfusion is calculated, and an unmanned aerial vehicle site serial number NSI corresponding to the TNS is recorded; and
(c) weighed triangle comprehensive evaluation is performed on the hospital NHI and the unmanned aerial vehicle site NSI, and a weighed triangle judgment index C is calculated as follows:
$$C = \frac{T N H}{\sqrt{2 * T N S * ( T N S + 0.5 * T S H )}} - 1$$
where TSH represents a road traffic time from the unmanned aerial vehicle site NSI to a hospital Q with shortest consuming time to the unmanned aerial vehicle site NSI;
when the index C is greater than 0, it is suggested transporting the patient to the unmanned aerial vehicle site NSI for the O-type red blood cell emergency blood transfusion, then transporting the patient to the hospital Q for further treatment, the blood use demand of the patient at the unmanned aerial vehicle site NSI is supplied by the hospital affiliated to the unmanned aerial vehicle site, and the blood use demand for further treatment is supplied by the hospital Q; and otherwise, it is suggested transporting the patient to the hospital NHI for O-type red blood cell emergency blood transfusion and further treatment, and blood use demand of the patient corresponds to the hospital NHI.

4. The emergency blood dispatching method based on early prediction and unmanned fast delivery according to claim 3, wherein in the step 4, said counting the total demand for the blood products in each hospital comprises:
denoting a number of all patients in a hospital i at time t as Nᵢ, comprising patients transported to the hospital i from the injury scene or the unmanned aerial vehicle site, and patients having emergency blood transfusion at the unmanned aerial vehicle site managed by the hospital i;
adopting, for the patient n, the staged multi-level emergency blood use prediction model to predict a category Ŷₙ, and performing calculation to obtain the number Rₙ of red blood cell blood product demands of the hospital i for the treatment of the patient n through Ŷₙ, a patient treatment route, and a patient blood type determination status;
wherein in the preliminary scheme, when Ŷₙ=0, Rₙ=0, and when Ŷₙ=1, determining whether an emergency blood product of a patient n is supplied by the hospital i; in case O-type red blood cell emergency blood transfusion is performed at the hospital or the unmanned aerial vehicle site managed by the hospital i, Rₙ=2, and in case the hospital i is not required to prepare the emergency blood product of the patient n, Rₙ=0;
wherein in the improved scheme, when Ŷₙ=0 , Rₙ=0, when Ŷₙ=1, determining whether a blood type of the patient n has been determined at time t; in case the blood type is not determined, Rₙ=0, in case the blood type has been determined, Rₙ=2, in case Ŷₙ=2, determining whether O-type red blood cells for emergency blood transfusion of the patient n are supplied by the hospital i, whether the specific blood-type red blood cells for further treatment are supplied by the hospital i, and whether the blood type of the patient n has been determined at time t; in case all the red blood cells of the patient n are supplied by the hospital i and the blood typed is not determined, Rₙ=2, in case all the red blood cells of the patient n are supplied by the hospital i and the blood type has been determined, Rₙ=4; in case for the patient n, only the O-type red blood cells are supplied by the hospital i, Rₙ=2; in case for the patient n, only the specific blood-type red blood cells are supplied by the hospital i and the blood type is not determined, Rₙ=0; and in case for the patient n, only the specific blood-type red blood cells are supplied by the hospital i and the blood type has been determined, Rₙ=2; and
gathering blood use demands of all the patients in the hospital i, and evaluating a total demand for the blood products at time t, wherein a total demand of the blood products of the hospital i at time t is Dᵢ=Σₙ₌₁^{N}^{ᵢ}Rₙ.

5. The emergency blood dispatching method based on early prediction and unmanned fast delivery according to claim 4, wherein in the step 4, said calculating the demand tension degree of all the blood products of all the patients in each hospital and performing ranking, so as to form the in-hospital blood product supply sequential order table comprises:
predicting the category Ŷₙ using the staged multi-level emergency blood use prediction model to for the patient n in the hospital i, calculating a blood use tension degree zₙ^{(patient)} of the patient n in the hospital i in combination with a duration of the patient n waiting for the blood product, and calculating a demand tension degree z_{n,p}^{(blood)}, p=1, . . . , Pₙ of all red blood cells of the patient n in the hospital i according to zₙ^{(patient)}, where Pₙ represents a total demand for the red blood cells of the patient n;
wherein in the preliminary scheme, when Ŷₙ=0 , zₙ^{(patient)}=0; when Ŷₙ=1, zₙ^{(patient)}=gₙ*AWTₙ, where gₙ represents whether the emergency blood product of the patient n is supplied by the hospital i, when the blood product is supplied by the hospital, gₙ=1, otherwise, gₙ=0, AWTₙ represents time spent by the patient n in waiting for the emergency blood product at time t; when Ŷₙ=0, a demand tension degree of the blood product z_{n,p}^{(blood)} is zero; and when Ŷₙ=1, the demand tension degree of the blood product z_{n,p}^{(blood)}=zₙ^{(patient)}, p=1,2;
wherein in the improved scheme, when Ŷₙ=0, zₙ^{(patient)}=0; when Ŷₙ=1, zₙ^{(patient)}=gₙ*AWTₙ, where gₙ represents whether the emergency blood product of the patient n is supplied by the hospital and whether the blood type has been determined; in case the emergency blood product is supplied by the hospital and the blood type has been determined, gₙ=1, and otherwise gₙ=0, AWTₙ represents the time spent by the patient n in waiting for the emergency blood product at time t; when Ŷₙ=2, zₙ^{(patient)}=A*(gₙ⁽¹⁾*AWTₙ⁽¹⁾+γ*gₙ⁽²⁾*AWTₙ⁽²⁾), where A represents a ratio coefficient of importance of blood transfusion for very emergent patients to importance of blood transfusion for moderate emergent patients, A>1, gₙ⁽¹⁾, gₙ⁽²⁾ represent whether the O-type red blood cell blood product for first emergency treatment of the patient n is supplied by the hospital, and whether the specific blood-type red blood cells for further treatment are supplied by the hospital and whether the blood type has been determined, respectively; in case the O-type red blood cell blood product for first emergency treatment is supplied by the hospital, gₙ⁽¹⁾=1, otherwise gₙ⁽¹⁾=0; and in case the specific blood-type red blood cells for further treatment are supplied by the hospital and the blood type has been determined, gₙ⁽²⁾=1, otherwise gₙ⁽²⁾=0, where AWTₙ⁽¹⁾, AWTₙ⁽²⁾ represent time spent by patient n in waiting for O-type red blood cells required for first emergency treatment at time t and time spent by the patient n in waiting for the specific blood-type red blood cells required for further treatment at time t, respectively, γ is a value discount factor of the specific blood-type red blood cells required for further treatment, and γ∈[0,1); when Ŷₙ=0, the demand tension degree for the blood z_{n,p}^{(blood)} is zero; when Ŷₙ=1, the demand tension degree for the blood product z_{n,p}^{(blood)}=zₙ^{(patient)}, p=1,2; and when Ŷₙ=2, the demand tension degree for the blood product z_{n,p}^{(blood)}=A*gₙ⁽¹⁾*AWTₙ⁽¹⁾, p=1,2, and z_{n,p}^{(blood)}=A*γ*gₙ⁽²⁾*AWTₙ⁽²⁾, p=3,4; and
performing ranking on all the blood products required by the hospital i in a descending order according to z_{n,p}^{(blood)}, and forming the in-hospital blood product supply sequential order table according to a rule of demand tension degree priority.

6. The emergency blood dispatching method based on early prediction and unmanned fast delivery according to claim 5, wherein the step 5 comprises:
step (5.1) measuring a supply and demand condition of blood products in each hospital, and building a current dispatching and delivery scheme according to delivery states of the transport tools;
wherein a blood product inventory in the hospital i is denoted as Iᵢ, and a number of in-transport blood products transported to the hospital i is denoted as Wᵢ;
$${W}_{i} = {\sum }_{u = 1}^{U} B U * ( I ( {SU}_{u} = i ) + {\sum }_{k = 1}^{C {U}_{u}} I ( {RU}_{u}^{k} = i ) ) + {\sum }_{t = 1}^{T} B T * ( I ( {ST}_{t} = i ) + {\sum }_{k = 1}^{C {T}_{t}} I ( {RT}_{t}^{k} = i ) )$$
where U and T represent a number of unmanned aerial vehicles and a number of blood delivery cars managed by a blood center, respectively, maximum loading quantities of the unmanned aerial vehicles and the blood delivery cars are BU and BT, respectively, and I(⋅) is an indicator function;
a set SU={SU₁, . . . , SUᵤ, . . . , SU_{U}} represents a condition of starting a unmanned aerial vehicle, where SUᵤ is valued as 0, i, or −i, representing that a uth unmanned aerial vehicle is in a standby state in the blood center, on the way to the hospital i, or on the way back to the blood center from the hospital i, respectively; CUᵤ represents a number of flights scheduled for the uth unmanned aerial vehicle; a set RUᵤ={RUᵤ¹, . . . , RUᵤ^{k}, . . . , RUᵤ^{CU}^{ᵤ}} represents a target hospital where the uth unmanned aerial vehicle is scheduled to fly; RUᵤ^{k}=i represents that a target hospital of a kth flight of the uth unmanned aerial vehicle scheduled to fly is the hospital i; and a set RU={RU₁, . . . , RUᵤ, . . . , RU_{U}};
a set ST={ST₁, . . . , STₜ, . . . , ST_{T}} represents a condition of starting a blood delivery car, STₜ is valued as 0, i, or −i, representing that a tth blood delivery car is in a standby state in the blood center, on the way to the hospital i, or on the way back to the blood center from the hospital i, respectively; CTₜ is the number of trips scheduled for the tth blood delivery car; a set RTₜ={RTₜ¹, . . . , RTₜ^{k}, . . . , RTₜ^{CT}^{ₜ}} represents a target hospital where the tth blood delivery car is scheduled to drive; RTₜ^{k}=i represents that the target hospital of a kth trip scheduled for the tth blood delivery car is the hospital i; and a set RT={RT₁, . . . , RTₜ, . . . , RT_{T}};
when a prepared blood volume of the hospital does not satisfy a demand blood volume Dᵢ, namely Iᵢ+Wᵢ<Dᵢ, the hospital i is marked to be in a blood-lacking state;
during initial dispatching, Wᵢ=0, all the unmanned aerial vehicles and all the blood delivery cars are in the standby state in the blood center; and
the sets SU, RU, ST, RT and the in-hospital blood product supply sequential order table of each hospital form a current dispatching and delivery scheme;
step (5.2) gathering all hospitals marked being in the blood-lacking state in a set LH, and obtaining LH={l₁, . . . , lⱼ, . . . , l_{N}_{^{(lack)}}}, where N^{(lack)} represents the number of hospitals in the blood-lacking state, and lⱼ represents a jth hospital in the blood-lacking state;
calculating, based on the current dispatching and delivery scheme, an overall future blood product supply tension degree estimated value zⱼ^{(hospital)} of the jth hospital in the blood-lacking state in the set LH as:
$${z}_{j}^{( hospital )} = {\sum }_{n = 1}^{{N}_{{l}_{j}}} {\sum }_{p = 1}^{{R}_{n}} {z}_{n , p}^{( estimated )}$$
where z_{n,p}^{(estimated)} represents a future supply tension degree estimated value of a pth unit of red blood cell blood products of the patient n according to the current dispatching and delivery scheme, and Nₗ_{ⱼ} represents a total number of patients of the jth hospital in the blood-lacking state;
selecting a hospital with a maximum value in all zⱼ^{(hospital)}, denoted as a hospital m, and preferably performing dispatching blood delivery for the hospital m;
step (5.3) working out a dispatching scheme with a waiting time of the hospital m being as short as possible based on the unmanned aerial vehicles and the blood delivery cars, comprising:
working out a next dispatching and delivery scheme by taking the minimum waiting time for the blood products of all the patients in the hospital m as a target using a cyclic sequence algorithm, based on the current dispatching and delivery scheme through ranking unmanned aerial vehicle priority, comparing the difference between the unmanned aerial vehicles and the blood delivery cars, and adjusting the indefinite-length route sequences, that is, sending a standby unmanned aerial vehicle to the hospital m, or adding a scheduled flight of the hospital m to a scheduled sequence of a certain unmanned aerial vehicle, or sending a standby blood delivery car to the hospital m, or adding a scheduled trip of the hospital m to a scheduled sequence of a certain blood delivery car;
firstly, calculating next flight ready time TNᵤ of an unmanned aerial vehicle u of the blood center, performing ascending ranking on TNᵤ, obtaining an unmanned aerial vehicle dispatching ranking table as UAVₗᵢₛₜ={K¹, K², . . . K^{U}}, and starting dispatching from an unmanned aerial vehicle K¹ with a minimum TNᵤ;
then, evaluating and determining dispatching strategy using a dispatching cost function, and comparing dispatching advantages of two tools by calculating dispatching cost differences of dispatching strategies of the unmanned aerial vehicles and the blood delivery cars;
sending an unmanned aerial vehicle K¹ with a shortest ready time to load a BU unit of blood products, and obtaining a dispatching cost value as J₁; sending the blood delivery car to load a BT unit of blood products, the BU unit of blood products being used for treating the patient, the remaining being wasted, and obtaining a dispatching cost value as J₂; calculating a dispatching cost difference DeltaJ=J₁−J₂, when DeltaJ<0, dispatching the unmanned aerial vehicle K¹, and otherwise, dispatching the blood delivery car with the shortest ready time; and
step (5.4) operating the steps (5.1) to (5.3) circularly until supply of the blood products of all the hospitals in the blood-lacking state is met.

7. The emergency blood dispatching method based on early prediction and unmanned fast delivery according to claim 6, wherein in the step 6, when a new trauma patient appears, the number of patients and the blood use demands for the patients in the step 2 are updated, and then the steps 3 to 5 are executed; when patient information changes, the blood use demands of the patients in the step 2 are updated, and then the steps 3 to 5 are executed; when the demands for the blood products the hospital change due to a change of a transport route of the patients and a blood type detection status of the patients, the demand for the blood products of the patients for the hospital is updated, and then the steps 4 to 5 are executed; when the unmanned aerial vehicle or the blood delivery car arrives at a certain hospital, the blood product inventory and the number of in-transport blood products of the hospital are updated, and then the steps 4 to 5 are executed; when the patients complete blood transfusion at the unmanned aerial vehicle site, the blood product inventory of the hospital affiliated to the unmanned aerial vehicle site and the blood product demands of the patients for the hospital affiliated to the unmanned aerial vehicle site are updated, and then the steps 4 to 5 are executed; and when the patients complete blood transfusion in a certain hospital, the blood product inventory of the hospital and the blood product demands of the patients for the hospital are updated, and then the steps 4 to 5 are executed.

8. An emergency blood dispatching system based on early prediction and unmanned fast delivery for implementing the method according to claim 1, comprising:
an emergency doctor terminal comprising an information input module and a first communication module; wherein the first communication module is configured to send patient information and receive emergency blood use prediction information of a patient and a recommended scheme of a transport destination of the patient; and
a dispatching command platform comprising a second communication module, a demand analysis monitoring module and a dispatching calculation module; wherein the second communication module is configured to receive patient information and send a blood supply demand and dispatching instructions; the demand analysis monitoring module is configured to determine an emergency blood use demand condition of the patient and comprehensively evaluate a demand blood volume of a hospital, an in-hospital inventory and an in-transport blood volume condition through an emergency blood use prediction model; and the dispatching calculation module is configured to generate the dispatching instructions of unmanned aerial vehicles and blood delivery cars, and send the dispatching instructions through the second communication module.
